(19) United States
(12) Patent Application Publication
(10) Pub. No.: US 2024/0395359 A1
(43) Pub. Date: Nov. 28, 2024

(54) REGION-AMBIGUOUS VARIANT DETECTION

(71) Applicant: Illumina, Inc., San Diego, CA (US)

(72) Inventors: Vitor Ferreira Onuchic, San Diego, CA (US); Shunhua Han, San Diego, CA (US)

(21) Appl. No.: 18/674,538

(22) Filed: May 24, 2024

Related U.S. Application Data
(60) Provisional application No. 63/469,319, filed on May 26, 2023.

(51) Int. Cl. G16B 20/20; G16B 20/10

(52) U.S. Cl. CPC G16B 20/20; G16B 20/10

(57) ABSTRACT

Methods, systems, and apparatus, including computer programs, for region-ambiguous joint detection. The method can include actions of obtaining a plurality of haplotypes, generating a plurality of joint diplotype candidates, wherein at least two of the plurality of haplotypes are associated with a first region of a reference genome and at least two of the other haplotypes are associated with a second region of the reference genome, the plurality of joint diplotype candidates comprising multiple different copy number configurations, determining a posterior probability for each of the plurality of joint diplotype candidates, determining a preferred copy number configuration of the multiple different copy number configurations, determining a region-ambiguous quality score for the plurality of joint diplotype candidates in a given position, and determining whether a variant exists in at least one of the first or second region of the reference genome based on the determined region-ambiguous quality score.

### CLAIM OF PRIORITY

This application claims priority under 35 USC § 119 (e) to U.S. Patent Application Ser. No. 63/469,319, filed on May 26, 2023, the entire contents of which are hereby incorporated by reference.

### FIELD

This specification generally relates to techniques for gene sequencing and comparison, e.g., of genomic data.

### BACKGROUND

Gene sequencing is a process that includes determining the order of nucleotides (A, C, G, and T) in a deoxyribonucleic acid (DNA) molecule. Instances of the nucleotide adenine in genomic data can be represented in a sequence by the letter “A.” Similarly, instances of nucleotides guanine, cytosine, thymine, or uracil in ribonucleic acid (RNA), can be represented by “G”, “C”, “T”, or “U”, respectively.

Genomic sequencing can be combined with genomic read mapping to identify the locus of a gene and the distances between genes. Computers can be used to analyze one or more sets of genomic data and correlate a collection of molecular markers, such as a series of nucleotides, with their respective positions on a given reference genome. In this way, a computer can be used to “map” the collection of molecular markers onto the reference genome.

### SUMMARY

The present disclosure is directed to systems, methods, apparatuses, computer programs, or any combination thereof, for determining a likelihood that a variant exists in the plurality of joint diplotype genotypes for at least one of region of a reference genome (e.g., a reference sequence). The accurate identification of a variant and the determination of the location in a genome from which the variant likely originated is important to accurately identify genetic differences that may be responsible for various phenotypic traits and diseases. For example, accurate identification of a variant and the location from which the variant likely originated can identify such variants as candidates to be used as biomarkers. Accordingly, accurate identification of variants and the location in a genome from which the variant likely originated can aid in understanding the genetic basis of traits and diseases and help identify potential targets for drug development.

However, the accurate identification of variants can be challenging, particularly in paralogous regions of the genome. Paralogous regions are regions of the genome that have high genotypic similarities, which can make it difficult to accurately identify the true source location of a read (e.g., a nucleic acid sequence). Variant calling typically uses alignment techniques to identify reads that come from a specific location and detect the underlying sequence at that location. However, if these techniques cannot confidently map a group of reads to a specific location, conventional variant callers may ignore them, even if the reads contain important information. Additionally, mismapped reads can result in detection errors. Short-read sequencing technologies are especially susceptible to these problems, and conventional detection often leaves large regions of the genome in the dark.

The limitations of conventional variant calling in the presence of paralogous regions have led to the development of new approaches that take into account the uncertainty surrounding the source location of reads. These approaches use a variety of techniques, such as pileup analysis and Bayesian modeling, to more accurately identify variants in paralogous regions.

For example, variant calling using quality scores can mitigate some of the problems with conventional variant calling. Conventional quality score variant calling can include Multi-Region Joint Detection (MRJD). Instead of considering each region in isolation like conventional variant calling, MRJD considers all locations from which a group of reads may have originated and attempts to detect the underlying sequences jointly using all available information to generate a quality score. However, existing MRJD approaches have limitations that will lower the variant detection sensitivity. One limitation is that existing MRJD approaches cannot genotype and report the variant if the variant exists but cannot be placed in any of the paralogous region with high confidence (e.g., a high quality score). Another limitation is that in regions impacted by gene conversion or a crossover event, existing MRJD approaches might assign the variant to the wrong paralogous copy.

Disclosed herein are systems, methods, apparatuses, computer programs, or any combination thereof for region-ambiguous variant detection. In this implementation, region-ambiguous variant detection provides improvements to conventional quality score variant calling (e.g., MRJD) in regions of a genome that have high genotypic similarities. Region-ambiguous variant detection determines and uses a new, region-ambiguous quality score that provides an indication whether a variant has been mapped to ambiguous regions or to the wrong region. Region-ambiguous variant detection utilizing this new, region-ambiguous quality score solves the problems associated with conventional variant calling described above by enabling greater sensitivity of variant calls, especially in regions that lack anchoring sites that help assign the variant to one of the paralogous copies, or in regions with gene conversion or a crossover event between paralogous copies.

This greater sensitivity afforded by the region-ambiguous variant detection of the present disclosure improves upon the conventional MRJD methods described above. Those conventional MRJD methods first compute the quality for a genotype with heterozygous or homozygous variants specific to a position in region 1. In these conventional MRJD methods, if a relative probability of joint diplotypes that support this genotype is similar to the probability of joint diplotypes that support homozygous reference alleles, a variant would not be called. In contrast, the region-ambiguous variant detection methods of the present disclosure enable greater sensitivity of the variant calls, especially in regions that lack anchoring sites that help assign the variant to one of the paralogous regions, or in regions with gene conversion or crossover events between paralogous regions. This greater sensitivity relative to conventional variant calling methods is shown in, for example, FIG. 4 of the present disclosure, which illustrates a variant recall rate of 95% (rounded to two digits) with a precision of 20%. This experimental data showing a high variant recall rate and low precision highlights the increased variant sensitivity achieved using the region-ambiguous variant detection of the present disclosure.

The region-ambiguous variant detection of the present disclosure provides an indication of whether a variant is present in either region 1 or region 2 of a set of paralogous regions. Using this novel region-ambiguous variant detection, a high region-ambiguous quality score for the variant being in either region 1 (j) or region 2 (i) can represent that a variant is present but ambiguously assigned. Region-ambiguous variant detection can report that there is a variant in both regions with a genotype (e.g., 0/0/0/1) that indicates a variant allele out of four total alleles (in both region 1 and region 2). Experimental data has shown that employing the region-ambiguous variant calling methods of the present disclosure on paralogous genes PMS2 and PMS2CL resulted in not only a higher recall rate of 100% for INDELs but also a higher precision of 57% (rounded to two digits) as shown in FIG. 5. Similarly, employing the region-ambiguous variant calling methods of the present disclosure on these same paralogous genes PMS2 and PMS2CL resulted in a higher recall rate of 90% (rounded to two digits) and also a higher precision of 97% (rounded two digits) for SNPs as shown in FIG. 5.

According to an innovative aspect of the present disclosure, a method for identifying variants using region-ambiguous variant detection in genetic sample sequences is disclosed. In one aspect, the method can include obtaining a plurality of haplotypes from one or more reads of a biological sample, generating a plurality of joint diplotype candidates comprising four or more haplotypes of the plurality of haplotypes, wherein at least two of the haplotypes of the plurality of haplotypes are associated with a first region of a reference genome and at least two other haplotypes of the plurality of haplotypes are associated with a second region of the reference genome, the plurality of joint diplotype candidates comprising multiple different copy number configurations, determining a posterior probability for each of the plurality of joint diplotype candidates, determining a preferred copy number configuration of the multiple different copy number configurations, wherein the preferred copy number configuration is a copy number configuration that occurs more frequently than any other copy number configuration in the plurality of joint diplotype candidates, determining a region-ambiguous quality score for the plurality of joint diplotype candidates in a given position in a given region based on a ratio of (i) the posterior probability associated with each joint diplotype candidate of the plurality of joint diplotype candidates having the preferred copy number configuration, and (ii) the posterior probability of a homozygous joint diplotype candidate of the plurality of joint diplotype candidates that only includes the reference allele at the given position and given region of the reference genome, and determining whether a variant exists in at least one of the first or second region of the reference genome based on the determined region-ambiguous quality score.

Other implementations of this and other aspects include corresponding systems, apparatus, and computer programs, configured to perform the actions of the methods, encoded on computer storage devices. A system of one or more computers can be so configured by virtue of software, firmware, hardware, or a combination of them installed on the system that in operation cause the system to perform the actions. One or more computer programs can be so configured by virtue of having instructions that, when executed by data processing apparatus, cause the apparatus to perform the actions.

The foregoing and other embodiments can each optionally include one or more of the following features, alone or in combination. For instance, in some implementations, actions include identifying the at least two different regions in the reference sequence as paralogous or homologous regions.

In some implementations, obtaining a plurality of haplotypes from one or more reads of a biological sample includes generating a plurality of joint diplotype candidates comprising four or more haplotypes of the plurality of haplotypes. For example, at least two of the haplotypes of the plurality of haplotypes can be associated with a first region of a reference genome and at least two other haplotypes of the plurality of haplotypes are associated with a second region of the reference genome, and the plurality of joint diplotype candidates can include multiple different copy number configurations. In some implementations determining a posterior probability for each of the plurality of joint diplotype candidates can include determining a preferred copy number configuration of the multiple different copy number configurations, wherein the preferred copy number configuration is a copy number configuration that occurs more frequently than any other copy number configuration in the plurality of joint diplotype candidates.

In some implementations, determining a region-ambiguous quality score for the plurality of joint diplotype candidates in a given position in a given region is based on a ratio of (i) the posterior probability associated with each joint diplotype candidate of the plurality of joint diplotype candidates having the preferred copy number configuration, and (ii) the posterior probability of a homozygous joint diplotype candidate of the plurality of joint diplotype candidates that only includes the reference allele at the given position and given region of the reference genome, and determining whether a variant exists in at least one of the first or second region of the reference genome based on the determined region-ambiguous quality score.

In some implementations, the copy number configurations of the multiple different copy number configurations are a copy number of each allele in the corresponding position of interest in the first region and the second region of the reference genome.

In some implementations, determining whether the variant exists in at least one region of the reference genome based on the determined region-ambiguous quality score includes comparing the region-ambiguous quality score to a predetermined threshold.

In some implementations, actions include determining that the region-ambiguous quality score satisfies the predetermined threshold based on comparing the region-ambiguous quality score to the predetermined threshold; and determining that an actual variant is likely to exist in at least one of the first region or the second region of the reference genome.

In some implementations, actions include determining that the region-ambiguous quality score does not satisfy the predetermined threshold based on comparing the region-ambiguous quality score to the predetermined threshold; and determining that an actual variant is not likely to exist in at least one of the first region or the second region of the reference genome.

In some implementations, determining the region-ambiguous quality score for the plurality of joint diplotype candidates includes determining the region-ambiguous quality score for the plurality of joint diplotype candidates based on (i) a sum of the posterior probability associated with each joint diplotype candidate of the plurality of the joint diplotype candidates having the preferred copy number configuration and (ii) the posterior probability of the homozygous joint diplotype of the plurality of joint diplotype candidates that matches the reference allele in each of the first region of the reference genome and the second region of the reference genome.

In some implementations, the first region and the second region of the reference genome are a subset of a quantity of regions of the reference genome. In some implementations, the quantity of regions of the reference genome is based on a quantity of paralogous regions located in the reference genome.

The details of one or more embodiments of the invention are set forth in the accompanying drawings and the description below. Other features and advantages of the invention will become apparent from the description, the drawings, and the claims.

### BRIEF DESCRIPTION OF THE DRAWINGS

FIG. 1 is a diagram showing an example of a system for region-ambiguous variant detection.

FIG. 2 is a flow diagram illustrating an example of a process for region-ambiguous variant detection.

FIG. 3 is a diagram illustrating an example of a computing system used for region-ambiguous variant detection.

FIG. 4 is a graph showing the benchmark on conventional germline variant calling, conventional MRJD, and region-ambiguous variant detection on SNP detection in in the high homology region of the PMS2 gene.

FIG. 5 is a graph showing region-ambiguous variant detection ability to detect alternative alleles in the paralogous genes PMS2 and PMS2CL.

Like reference numbers and designations in the various drawings indicate like elements.

### DETAILED DESCRIPTION

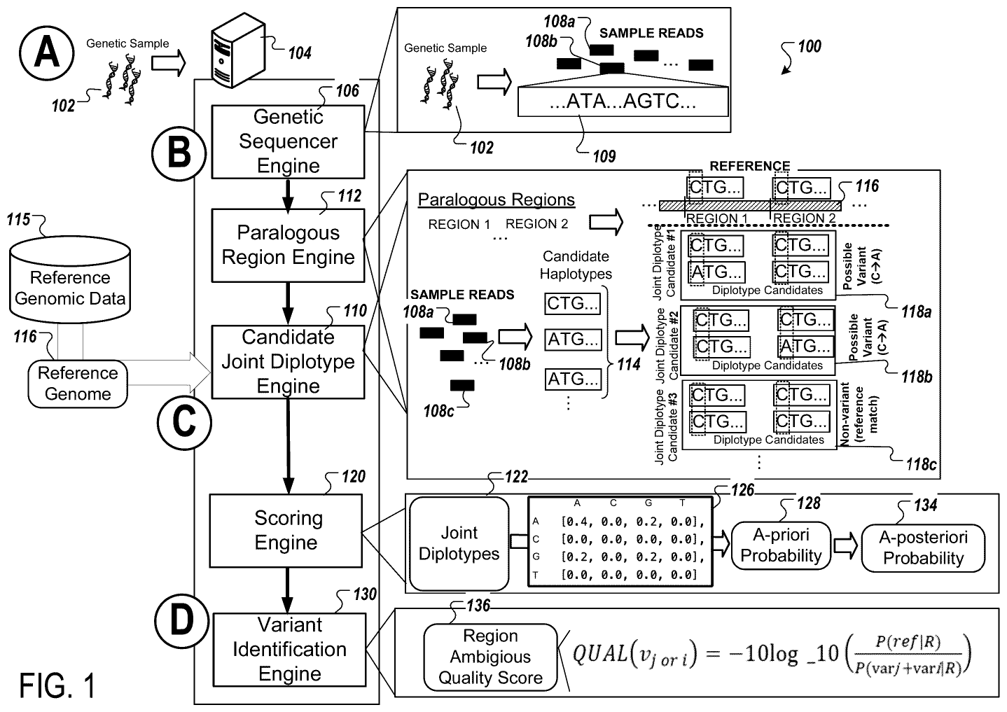

FIG. 1 is a diagram showing an example of a system 100 for region-ambiguous variant detection. The system 100 includes a control unit 104 and database 115. The control unit 104 can include, or be communicably connected to, one or more processing devices to perform operations described in reference to the control unit 104. The control unit 104 can access data stored on the database 115, such as reference genome 116.

In general, FIG. 1 shows candidate variants being identified and processed to determine if any of the candidate variants are actual variants that are in one or more regions. FIG. 1 is described with reference to stages A through D. Although described in order from stage A through D, operations of the system 100 can occur in different orders or in parallel. For example, the system 100 can engage in one or more parallel processing operations, such as identifying paralogous regions and mapping one or more candidate variants to one or more paralogous regions. In some implementations, the control unit 104 identifies one or more paralogous regions or sample sequences for processing.

In stage A, the control unit 104 obtains genetic sample data. The genetic sample data can be a genetic sample from an organism, such as a dog, human, or the like. The genetic sample can indicate DNA or RNA of the organism. The control unit 104 can be used to identify genetic variants with confidence values in the given organism based on detected variants in the genetic sequence.

In stage B, the control unit 104 processes the genetic sample 102. For example, a genetic sequencer engine 106 of the control unit 104 can sequence the genetic sample 102 to generate a sequence list of bases present in the DNA of the genetic sample 102. Sample reads 108a-c are shown in FIG. 1. In general, the genetic sequencer engine 106 can generate hundreds or thousands of sequences. An example sequence stretch of sample read 108b is shown in item 109 as “ . . . ATA . . . AGTC . . . . ” representing the identification, by the genetic sequencer engine 106, of a sequence of adenine followed by thymine followed by adenine and adenine followed by guanine followed by thymine followed by cytosine.

The genetic sequencer engine 106 can be included in the control unit 104 or be communicably connected to the control unit 104. In some implementations, the control unit 104 includes one or more processors that provide or obtain data from a genetic sequencer machine configured to perform next-generation “short-read” or third-generation “long-read” sequencing methods, among others.

The control unit 104 identifies one or more paralogous regions. For example, the control unit 104 can operate a paralogous region engine 112 that identifies one or more paralogous regions. In some implementations, the control unit 104 identifies paralogous regions in a reference genome, such as a reference genome 116 obtained from the database 115. For example, the control unit 104 can compare one or more sequences of the reference genome and determine sequences that satisfy one or more similarity thresholds as paralogous or homologous regions. In some implementations, the database 115 includes one or more processors communicably connected to one or more electronic storage devices configured to store digital data.

In some implementations, the paralogous region engine 112 obtains input from a user. For example, the paralogous region engine 112 can obtain input from a user indicating specific paralogous or homologous regions. Genes where the paralogous region engine 112 identifies paralogous or homologous regions can include PMS2 or PMS2CL among other genes.

In some implementations, the control unit 104 identifies paralogous regions using base sequences generated from the genetic sample 102. For example, the paralogous region engine 112 can compare one or more regions from one or more sequenced portions of the genetic sample 102. The paralogous region engine 112 can identify regions that satisfy one or more similarity thresholds.

In general, determining whether or not one or more regions, either in the sample or reference genome, satisfy one or more similarity thresholds can include one or more of determining one or more same bases are in the same location in multiple regions, above a percentage number of same bases are in the same location, among other methods.

In some implementations, the candidate joint diplotype engine 110 obtains a reference genome 116. For example, the candidate joint diplotype engine 110 can obtain the reference genome 116 from the database 115 for reference genomic data. The reference genome 116 can indicate fully or partially sequenced genetic material. The reference genome 116 can include reference bases in multiple positions within multiple genes. Deviation from the reference genome 116 at any given position can be considered a variant (e.g., a sample sequence from the genetic sample 102 can match the reference 116 in positions 1-5 along the genetic sequence of the reference 116). However, in position 6, the reference 116 can indicate adenine where the sample sequence indicates thymine or some other nucleotide. If the sample is correctly mapped to this portion of the reference, e.g., based on positions 1-5 matching the reference, the sample sequence has a variant at the position 6.

Because the identified homologous or paralogous regions are identical or highly similar (e.g., differing in only one or two bases for a given sequence length), mapping, especially short read sequences, to these regions can be difficult. The control unit 104 helps to solve this problem by generating joint diplotype candidates from haplotypes that have been identified as potentially being mapped to one or more of the homologous or paralogous regions. Each joint diplotype candidate can include at least one candidate diplotype. Each candidate diplotype can include two or more haplotypes combined to form the candidate diplotype.

In the example of FIG. 1, the candidate joint diplotype engine 110 generates joint diplotype candidates #1 118a, #2 118b, and #3 118c, among others. A joint diplotype candidate generated by the joint diplotype engine 110 comprises four or more candidate haplotypes 114. A diplotype comprises two haplotypes paired together. Diplotypes in all paralogous regions form the joint diplotype candidates. Joint diplotype candidate #1 118a is an example of a candidate diplotype. For example, joint diplotype candidate #1 118a comprises four candidate haplotypes 114, two in each region. Joint diplotype candidate #1 is a pairing of haplotype “CTG . . . ” with haplotype “ATG . . . ” in the first region of reference genome 116 which forms a first diplotype; and haplotype “CTG . . . ” with haplotype “CTG . . . ” in the second region of the reference genome 116 forms a second diplotype. Joint diplotype candidate #2 118b is another example of a candidate diplotype. Joint diplotype candidate #2 is a pairing of haplotype “CTG . . . ” with haplotype “ATG . . . ” in the second region of reference genome 116 which forms a first diplotype and haplotype “CTG . . . ” with haplotype “CTG . . . ” in the first region of the reference genome 116 which formed a second diplotype. As shown in FIG. 1, joint diplotype candidate #3 118c is a pairing of haplotype “CTG . . . ” with haplotype “CTG . . . ” which matches the reference genome 116 regardless of region 1 (e.g., a first diplotype) or region 2 (e.g., a second diplotype). The plurality of joint diplotype candidates 118 comprising multiple different copy number configurations represents the copy number of each of the alleles in all paralogous regions.

Joint diplotype candidate #1 118a supports a potential variant in the first position at REGION 1 because the first candidate diplotype at the first position includes C and A where the reference is C. Because the joint diplotype candidate #1 118a includes an A instead of C, the first candidate diplotype can be a potential variant with a copy number configuration of CACC. Joint diplotype candidate #2 118b supports a potential variant in the first position at REGION 2 because the joint diplotype candidate #2 118b at the first position of REGION 2 includes C and A where the reference is C. Because the joint diplotype candidate #2 includes C and A instead of C, the joint diplotype candidate #2 can be a potential variant with a copy number configuration of CCCA. Joint diplotype candidate #3 118c is a non-variant candidate where no positions support a variant with a copy number configuration of CCCC.

In general, the candidate joint diplotype engine 110 can generate joint diplotype candidates for as many regions that are identified as homologous or paralogous. In some implementations, the candidate joint diplotype engine 110 generates joint diplotype candidates that include hundreds or thousands of candidate diplotypes mapped to the hundreds or thousands of paralogous or homologous regions. In some implementations, the candidate joint diplotype engine 110 randomly permutes the identified candidate haplotypes for all identified regions until all permutations are generated. The set of all joint diplotype candidates can include a full set of all possible permutations of the identified candidate haplotypes mapped to the identified regions.

The question can be presented, which candidates correspond to actual variants to the reference genome 116 regardless of region? It is possible that either joint diplotype candidate #1 118a or joint diplotype candidate #2 118b is the correct mapping. Since the posterior probability of joint diplotype candidate #1 118a is equal to joint diplotype candidate #2 118b, previous solutions (e.g., MRJD) would overlook the existence of a variant at all. A determination of a region non-specific variant can be helpful when used as the basis for treatment or medical predictions. One important reason for having this type of variant reported is in clinical diagnostic settings where identifying all potential pathogenic variants is paramount. Reporting a region non-specific variant can indicate that confirmatory testing is required (e.g., long-range PCR-based tests); maximizing recall is preferred, as long as the false positive rate is not excessive.

In stage C, the control unit 104 generates prior probability values to help determine which of the joint diplotype candidates are likely to be correct. In general, scoring engine 120 of the control unit 104 can obtain the diplotypes 122 for each joint diplotype candidate 118a, 118b, and 118c. For example, the scoring engine 120 can generate an a-prior probability for each diplotype 122 within a joint diplotype candidate using diplotype probability matrix 126, and generate an a-priori probability 128 for a joint diplotype candidate. In this implementation, the scoring engine 120 of the control unit 104 can determine a posterior probability (i.e., a-posteriori probability) 134 for each of the plurality of joint diplotype candidates 118a, 118b, and 118c.

N can represent the number of regions to be jointly processed (e.g., REGION 1 and REGION 2), Hₖ can represent candidate haplotypes (e.g., candidate haplotypes 114) where k=1 . . . K and each can include various SNPs, insertions and/or deletions relative to a reference sequence (e.g., the reference genome 116), Gₘ can represent candidate solutions for both phases ø=1,2 and all regions n=1 . . . N (e.g., joint diplotype candidate #1 118a), G_{m,ø,n} can be a candidate solution generated from the set of candidate haplotypes {H₁ . . . Hₖ}, and rᵢ can represent paired reads {r_{i,1}, r_{i,2}}. The probabilities for each read are calculated for each candidate haplotype (Hₖ) haplotype P(rᵢ|Hₖ), for example, by using a Hidden Markov Model (HMM). In the case of datasets with paired reads, rᵢ indicates the pair {r_{i,1}, r_{i,2}}, and P(rᵢ|Hₖ)═P(r_{i,1}|Hₖ) P(r_{i,2}|Hₖ). In the case of datasets with linked reads (e.g., barcoded reads), rᵢ indicates the group of reads {r_{i,1} . . . r_{i,NL}} that came from the same long molecule, and P(rᵢ|Hₖ)=Πₙ₌₁^{NL} P(ri,n|Hk). For each candidate solution Gₘ, m=1 . . . M, the conditional probability of each read

$$P ( {r}_{i} \vert {G}_{m} ) = \frac{1}{2 N} {\sum }_{n = 1}^{N} {\sum }_{ϕ = 1}^{2} P ( {r}_{i} \vert {G}_{m , ϕ , n} )$$

is determined and conditional probability of the entire pileup R═{r₁ . . . r_{NR}}: P(R|Gₘ)=Πᵢ₌₁^{NR} P(ri|Gₘ).

In stage D, the control unit 104 determines whether the genetic sequence of the genetic sample 102 supports one or more variants. For example, a variant identification engine 130 of the control unit 104 can use the a-priori probability 128 generated by the scoring engine 120 to generate an a-posteriori probability 134. The a-posteriori probability 134 is calculated of each joint diplotype candidate given the observed pileup: P(Gₘ|R)=P(R|Gm)P(Gm)/Σᵢ₌₁^{M} P(R|Gi)P(Gi) where P(Gₘ) indicates the a-priori probability of the candidate solution. The control unit 104 can use the a-posteriori probability 134 to generate one or more values indicating the likelihood of one or more of the generated joint diplotype candidates being correct for one or more of the regions.

For example, the variant identification engine 130 of the control unit 104 can determine a preferred copy number configuration of the joint diplotype candidates 118a, 118b, 118c. A preferred copy number configuration is the most frequently occurring joint diplotype candidate configuration. In the example illustrated by FIG. 1, the preferred copy number configuration is the combination of the copy number configurations of joint diplotype candidate #1 (CACC) and joint diplotype candidate #2 (CCCA) because they both support the variant having three “C” and one “A”. This would give the genotype of 0/0/0/1 because there is one allele difference in the position as compared to the reference (e.g., C/C/C/A). In the example of FIG. 1 where there is one allele change in the position, the number of potential copy number configurations is five (e.g., C/C/C/C (i.e., 0/0/0/0), C/C/C/A (i.e., 0/0/0/1), C,C,A,A (i.e., 0/0/1/1), C,A,A,A (i.e., 0,1,1,1), and A,A,A,A (i.e., 1,1,1,1). In this implementation, a quality score in region 1 would be determined based on the likelihood ratio between preferred copy number configuration C,C,C,A and homozygous reference copy number configuration C/C/C/C. Likewise for region 2, a quality score in region 2 would be determined based on the likelihood ratio between preferred copy number configuration C,C,C,A and homozygous reference copy number configuration C/C/C/C.

For example, the variant identification engine 130 of the control unit 104 can determine a region-ambiguous quality score for the plurality of joint diplotypes based on (i) a sum of the first scores associated with each joint diplotype candidate having the preferred copy number configuration (e.g., three “C” and one “A”) and (ii) the first score of a homozygous joint diplotype in the plurality of joint diplotypes that matches the reference allele in each of the at least two different regions of the reference sequence (e.g., candidate joint diplotype candidate #3). The variant identification engine 130 can generate a region-ambiguous quality score 136. The region-ambiguous quality score 136 can determine the quality score of the preferred copy number configuration.

In another implementation, a quality score can be determined based on the joint diplotype candidate having a copy number configuration that matches the homozygous reference copy number configuration. In the example, illustrated by FIG. 1, the variant identification engine 130 of the control unit 104 can determine a copy number configuration of one or more of the joint diplotype candidates that match the homozygous reference copy number configuration. In this example illustrated by FIG. 1, the homozygous reference copy number configuration is C/C/C/C. In this implementation, the quality score is determined based on the homozygous reference copy number configuration is C/C/C/C. For example, if a posterior probability of the joint diplotype candidates that match the homozygous reference copy number configuration (e.g., C/C/C/C) is higher than the determined posterior probabilities determined for the other copy number configurations (e.g., C/C/C/A (i.e., 0/0/0/1), C,C,A,A (i.e., 0/0/1/1), C,A,A,A (i.e., 0,1,1,1), and A,A,A,A (i.e., 1,1,1,1), then a region-ambiguous quality score can indicate that an actual variant is not likely to exist.

In some implementations, the variant identification engine 130 determines that the region-ambiguous quality score 136 includes one or more values indicating a likelihood of a variant being present in either paralogous region. For example, a region-ambiguous quality score 136 can be reported as

$$QUAL ( {v}_{j or i} ) = - 10 log\_ 10 ( \frac{P ( ref \vert R )}{P ( var j + var i \vert R )} ) .$$

In the example illustrated in FIG. 1, P(ref|R) is the posterior probability of joint diplotype candidate #3 118c which is a non-variant candidate where no positions support a variant, and P(varj) is posterior probability of joint diplotype candidate #1 118a which supports the variant in region 1, and P(vari) is the posterior probability of joint diplotype candidate #2 118b which supports the variant in region 2. In some implementations, the variant identification engine 130 determines whether a variant exists in the plurality of joint diplotype genotypes for at least one of region of a reference sequence.

In another implementation, the variant identification engine 130 determines that the region-ambiguous quality score 136 does not include one or more values indicating a likelihood of variant being present in either paralogous region. For example, a region-ambiguous quality score 136 can be reported as QUAL(v_{j or i})=−10 log_10(P(ref|R). In this implementation, P(ref|R) is the posterior probability of a joint diplotype candidate which is a non-variant candidate where no positions support a variant (e.g., joint diplotype candidate #3 118c). If the posterior probability P(ref|R) of the joint diplotype candidates that match the homozygous reference copy number configuration (e.g., C/C/C/C) is higher than the determined posterior probabilities determined for the other copy number configurations (e.g., C/C/C/A (i.e., 0/0/0/1), C,C,A,A (i.e., 0/0/1/1), C,A,A,A (i.e., 0,1,1,1), and A,A,A,A (i.e., 1,1,1,1), then a region-ambiguous quality score can indicate that an actual variant is not likely to exist.

In some implementations, the variant identification engine 130 can determine that one or more values representing preferred copy number configuration are greater than or equal to one or more other values representing other generated joint diplotype candidates. In some implementations, the variant identification engine 130 ranks one or more values to determine a candidate with a highest value as the correct candidate. Data of the region-ambiguous quality score 136 can indicate a variant likeliness in either/any position or gene. The score provided by the region-ambiguous quality score 136 can be reported to a user. For example, region-ambiguous quality score 136 can be an output as a Variant Call Format (VCF) file or using other file formats. In this way, region-ambiguous variant detection can identify a quality score that determines that variants have been mapped to ambiguous regions. In some implementations, region-ambiguous variant detection can enable greater sensitivity in detection of variants in regions that lack anchoring sites or in regions that lack crossover or conversion events between paralogous regions.

Previous solutions (e.g., MRJD) first compute the quality for a genotype with heterozygous or homozygous variants specific to a position in region 1. If a relative probability of joint diplotypes that support this genotype is similar to the probability of joint diplotypes that support homozygous reference alleles, a variant would not be called. In contrast, region-ambiguous variant detection methods enable greater sensitivity of the variant calls, especially in regions that lack anchoring sites that help assign the variant to one of the paralogous regions, or in regions with gene conversion or crossover events between paralogous regions. Using this novel region-ambiguous variant detection, a high quality score for the variant being in either region 1 (j) or region 2 (i) can represent that a variant is present but ambiguously assigned. Region-ambiguous variant detection can report that there is a variant in both regions with a genotype (e.g., 0/0/0/1) that indicates a variant allele out of four total alleles (in both region 1 and region2). In some embodiments, additional tags in the INFO of the VCF file can indicate the variant is ambiguously placed. In some implementations, the control unit 104 generates one or more outputs using the expressions referenced in this document to identify whether or not a variant is a variant in either region 1 or region 2 (e.g., using the variant identification engine 130).

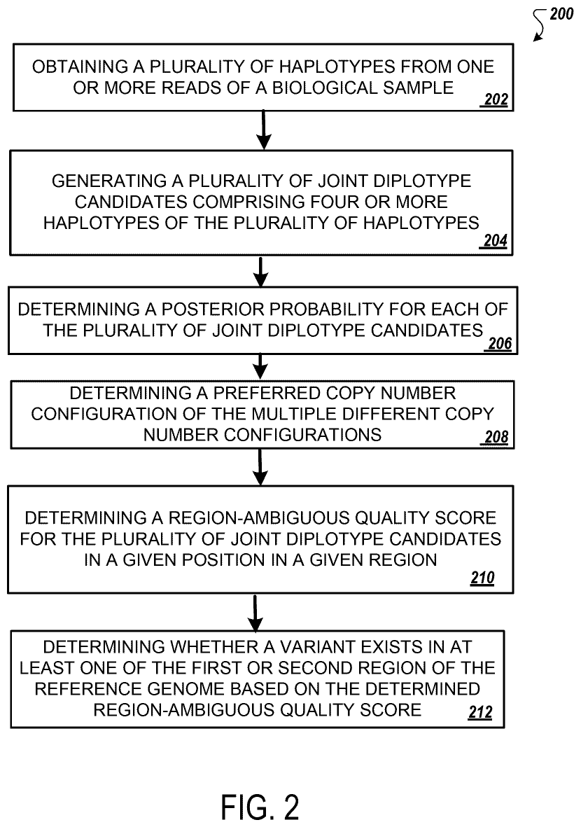

FIG. 2 is a flow diagram illustrating an example of a process 200 for region-ambiguous variant detection. The process 200 may be performed by one or more electronic systems, for example, the system 100 of FIG. 1.

The process 200 includes obtaining a plurality of haplotypes from one or more reads of a biological sample (202). For example, the control unit 104 can obtain, from the one or more sample reads 108a-b, one or more candidate haplotypes 114.

The process 200 includes generating a plurality of joint diplotype candidates comprising four or more haplotypes of the plurality of haplotypes (204). In this implementation, at least two of the haplotypes of the plurality of haplotypes are associated with a first region of a reference genome and at least two other haplotypes of the plurality of haplotypes are associated with a second region of the reference genome, the plurality of joint diplotype candidates comprising multiple different copy number configurations. In some implementations, copy number configurations of the multiple different copy number configurations are a copy number of each allele in the corresponding position of interest in the first region and the second region of the reference genome.

For example, the control unit 104 can generate diplotype candidates (e.g., including C and A in REGION 1 and C and C in REGION 2, among other nucleotides). For example, joint diplotype candidate #1 118a comprises four candidate haplotypes 114, two in each region. Joint diplotype candidate #1 is a pairing of haplotype “CTG . . . ” with haplotype “ATG . . . ” in the first region of reference genome 116 which forms a first diplotype; and haplotype “CTG . . . ” with haplotype “CTG . . . ” in the second region of the reference genome 116 forms a second diplotype. Joint diplotype candidate #2 118b is another example of a candidate diplotype. Joint diplotype candidate #2 is a pairing of haplotype “CTG . . . ” with haplotype “ATG . . . ” in the second region of reference genome 116 which forms a first diplotype and haplotype “CTG . . . ” with haplotype “CTG . . . ” in the first region of the reference genome 116 which formed a second diplotype. As shown in FIG. 1, joint diplotype candidate #3 118c is a pairing of haplotype “CTG . . . ” with haplotype “CTG . . . ” which matches the reference genome 116 regardless of region 1 (e.g., a first diplotype) or region 2 (e.g., a second diplotype).

The process 200 includes determining a posterior probability for each of the plurality of joint diplotype candidates (206). For example, the scoring engine 120 can generate an a-priori probability for each diplotype 122 within a joint diplotype candidate using diplotype probability matrix 126, and generate an a-priori probability 128 for a joint diplotype candidate. In this implementation, the scoring engine 120 of the control unit 104 can determine a posterior probability (i.e., a-posteriori probability) 134 for each of the plurality of joint diplotype candidates 118a, 118b, and 118c.

The process 200 includes determining a preferred copy number configuration of the multiple different copy number configurations (208). For example, determining a preferred copy number configuration includes determining the most frequently occurring joint diplotype candidate configuration. In the example illustrated by FIG. 1, the preferred copy number configuration is the combination of the copy number configurations of joint diplotype candidate #1 (CACC) and joint diplotype candidate #2 (CCCA) because they both support the variant having three “C” and one “A”. This would give the genotype of 0/0/0/1 because there is one allele difference in the position as compared to the reference (e.g., C/C/C/A). In the example of FIG. 1 where there is one allele change in the position, the number of potential copy number configurations is five (e.g., C/C/C/C (i.e., 0/0/0/0), C/C/C/A (i.e., 0/0/0/1), C,C,A,A (i.e., 0/0/1/1), C,A,A,A (i.e., 0,1,1,1), and A,A,A,A (i.e., 1,1,1,1). In this implementation, a quality score in region 1 would be determined based on the likelihood ratio between preferred copy number configuration C,C,C,A and homozygous reference copy number configuration C/C/C/C. Likewise for region 2, a quality score in region 2 would be determined based on the likelihood ratio between preferred copy number configuration C,C,C,A and homozygous reference copy number configuration C/C/C/C.

The process 200 includes determining a region-ambiguous quality score for the plurality of joint diplotype candidates in a given position in a given region (210). In some implementations, the variant identification engine 130 of the control unit 104 can determine a region-ambiguous quality score for the plurality of joint diplotype candidates in a given position in a given region based on a ratio of (i) the posterior probability associated with each joint diplotype candidate of the plurality of joint diplotype candidates having the preferred copy number configuration, and (ii) the posterior probability of a homozygous joint diplotype candidate of the plurality of joint diplotype candidates that only includes the reference allele at the given position and given region of the reference genome.

For example, the variant identification engine 130 of the control unit 104 can determine a region-ambiguous quality score for the plurality of joint diplotypes based on (i) a sum of the first scores associated with each joint diplotype candidate having the preferred copy number configuration (e.g., three “C” and one “A”) and (ii) the first score of a homozygous joint diplotype in the plurality of joint diplotypes that matches the reference allele in each of the at least two different regions of the reference sequence (e.g., candidate joint diplotype candidate #3). The variant identification engine 130 can generate a region-ambiguous quality score 136. The region-ambiguous quality score 136 can determine the quality score of the preferred copy number configuration.

In some implementations, the variant identification engine 130 of the control unit 104 can determine the region-ambiguous quality score for the plurality of joint diplotype candidates based on (i) a sum of the posterior probability associated with each joint diplotype candidate of the plurality of the joint diplotype candidates having the preferred copy number configuration and (ii) the posterior probability of the homozygous joint diplotype of the plurality of joint diplotype candidates that matches the reference allele in each of the first region of the reference genome and the second region of the reference genome.

In another implementation, a quality score can be determined based on the joint diplotype candidate having a copy number configuration that matches the homozygous reference copy number configuration. In the example, illustrated by FIG. 1, the variant identification engine 130 of the control unit 104 can determine a copy number configuration of one or more of the joint diplotype candidates that match the homozygous reference copy number configuration. In this example illustrated by FIG. 1, the homozygous reference copy number configuration is C/C/C/C. In this implementation, the quality score is determined based on the homozygous reference copy number configuration of C/C/C/C. For example, if a posterior probability of the joint diplotype candidates that match the homozygous reference copy number configuration (e.g., C/C/C/C) is higher than the determined posterior probabilities determined for the other copy number configurations (e.g., C/C/C/A (i.e., 0/0/0/1), C,C,A,A (i.e., 0/0/1/1), C,A,A,A (i.e., 0,1,1,1), and A,A,A,A (i.e., 1,1,1,1), then a region-ambiguous quality score can indicate that an actual variant is not likely to exist.

The process 400 includes determining whether a variant exists in at least one of the first or second region of the reference genome based on the determined region-ambiguous quality score (212). The variant identification engine 130 determines that the region-ambiguous quality score 136 includes one or more values indicating a likelihood of variant being present in either paralogous region. For example, a region-ambiguous quality score 136 can be reported as

$$QUAL ( {v}_{j or i} ) = - 10 log\_ 10 ( \frac{P ( ref \vert R )}{P ( var j + var i \vert R )} ) .$$

In the example illustrated in FIG. 1, P(ref|R) is the posterior probability of joint diplotype candidate #3 118c which is a non-variant candidate where no positions support a variant, and P(varj) is posterior probability of joint diplotype candidate #1 118a which supports the variant in region 1, and P(vari) is the posterior probability of joint diplotype candidate #2 118b which supports the variant in region 2. In some implementations, the variant identification engine 130 determines whether a variant exists in the plurality of joint diplotype genotypes for at least one of region of a reference sequence.

In some implementations, the variant identification engine 130 determines whether the variant exists in at least one region of the reference genome based on the determined region-ambiguous quality score by comparing the region-ambiguous quality score to a predetermined threshold. In another implementation the variant identification engine 130 determines that the region-ambiguous quality score satisfies the predetermined threshold based on comparing the region-ambiguous quality score to the predetermined threshold; and determining that an actual variant is likely to exist in at least one of the first region or the second region of the reference genome. In yet another implementation, the variant identification engine 130 determines that the region-ambiguous quality score does not satisfy the predetermined threshold based on comparing the region-ambiguous quality score to the predetermined threshold; and determining that an actual variant is not likely to exist in at least one of the first region or the second region of the reference genome.

In another implementation, the variant identification engine 130 determines that the region-ambiguous quality score 136 does not include one or more values indicating a likelihood of a variant being present in either paralogous region. For example, a region-ambiguous quality score 136 can be reported as QUAL(v_{j or i})=−10 log_10 (P(ref|R). In this implementation, P(ref|R) is the posterior probability of joint diplotype candidate which is a non-variant candidate where no positions support a variant (e.g., joint diplotype candidate #3 118c). If the posterior probability P(ref|R) of the joint diplotype candidates that match the homozygous reference copy number configuration (e.g., C/C/C/C) is higher than the determined posterior probabilities determined for the other copy number configurations (e.g., C/C/C/A (i.e., 0/0/0/1), C,C,A,A (i.e., 0/0/1/1), C,A,A,A (i.e., 0,1,1,1), and A,A,A,A (i.e., 1,1,1,1), then a region-ambiguous quality score can indicate that an actual variant is not likely to exist.

Multiple technological improvements and advantages of the present disclosure have been provided herein. However, the present disclosure is not limited to those improvements and advantages. Instead, a person of ordinary skill in the are would recognize many other technological improvements and advantages that result from the region-ambiguous variant calling methods, the entire list cannot be fully enumerated. Examples of these other technological improvements or advantages include accurate detection of variants relative to paralogous regions of a reference genome requiring performance of the operations described herein with reference to hundreds, thousands, millions, or tens of millions reference sequence locations. In some cases, the present disclosure enables performance of operations that could not reasonably or practicably be performed in a human mind in a reasonable amount of time given the complexity of the operations performed for set of, e.g., tens of millions of reference sequence locations.

As another example, the improvements to variant call accuracy achieved by the present disclosure reduce downstream processing that needs to be performed and improve the accuracy thereof. For example, a downstream process such as tertiary analysis performed using the variants identified by the present disclosure are more accurate and efficient, as they are operation on more accurate variant sets. Tertiary analysis on these more accurate variant sets can be executed and the results trusted in a more routine manner than conventional tertiary analysis operations that may be executed on less accurate variant sets, which can lead to analysis that needs to be performed again, results that cannot be trusted as much as those results generated by the present disclosure, or a combination of both.

Persons of ordinary skill in the art would recognize these, and other, improvements and advantages that naturally follow from implantation of the region-ambiguous variant calling methods described here.

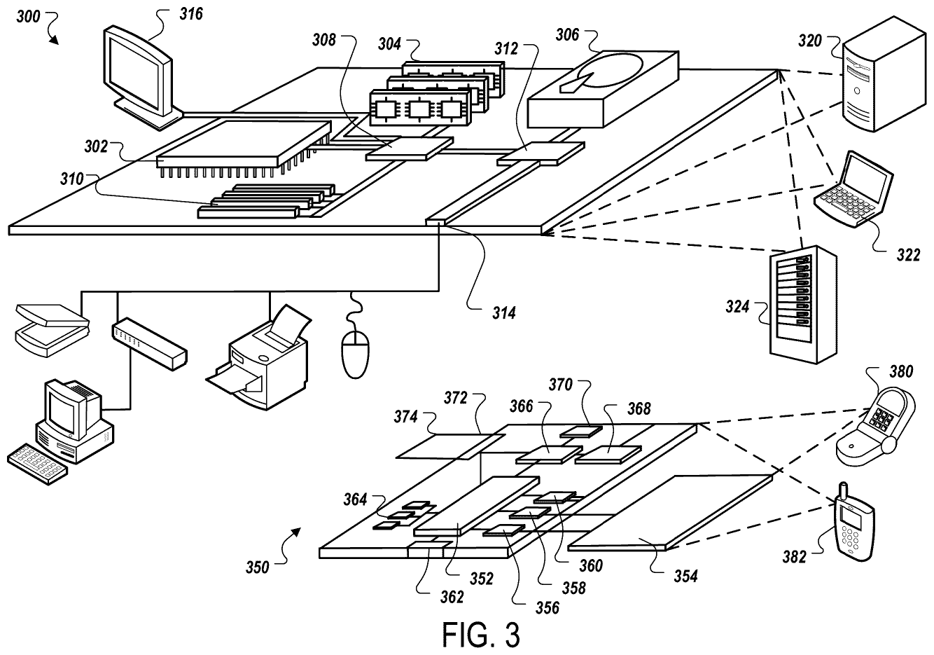

FIG. 3 is a diagram illustrating an example of a computing system used for multi-region joint detection. The computing system includes computing device 300 and a mobile computing device 350 that can be used to implement the techniques described herein. For example, one or more components of the system 100 could be an example of the computing device 300 or the mobile computing device 350, such as a computer system implementing the control unit 104 or various engines or models included therein or communicably connected to.

The computing device 300 is intended to represent various forms of digital computers, such as laptops, desktops, workstations, personal digital assistants, servers, blade servers, mainframes, and other appropriate computers. The mobile computing device 350 is intended to represent various forms of mobile devices, such as personal digital assistants, cellular telephones, smart-phones, mobile embedded radio systems, radio diagnostic computing devices, and other similar computing devices. The components shown here, their connections and relationships, and their functions, are meant to be examples only, and are not meant to be limiting.

The computing device 300 includes a processor 302, a memory 304, a storage device 306, a high-speed interface 308 connecting to the memory 304 and multiple high-speed expansion ports 310, and a low-speed interface 312 connecting to a low-speed expansion port 314 and the storage device 306. Each of the processor 302, the memory 304, the storage device 306, the high-speed interface 308, the high-speed expansion ports 310, and the low-speed interface 312, are interconnected using various busses, and may be mounted on a common motherboard or in other manners as appropriate. The processor 302 can process instructions for execution within the computing device 300, including instructions stored in the memory 304 or on the storage device 306 to display graphical information for a GUI on an external input/output device, such as a display 316 coupled to the high-speed interface 308. In other implementations, multiple processors and/or multiple buses may be used, as appropriate, along with multiple memories and types of memory. In addition, multiple computing devices may be connected, with each device providing portions of the operations (e.g., as a server bank, a group of blade servers, or a multi-processor system). In some implementations, the processor 302 is a single threaded processor. In some implementations, the processor 302 is a multi-threaded processor. In some implementations, the processor 302 is a quantum computer.

The memory 304 stores information within the computing device 300. In some implementations, the memory 304 is a volatile memory unit or units. In some implementations, the memory 304 is a non-volatile memory unit or units. The memory 304 may also be another form of computer-readable medium, such as a magnetic or optical disk.

The storage device 306 is capable of providing mass storage for the computing device 300. In some implementations, the storage device 306 may be or include a computer-readable medium, such as a floppy disk device, a hard disk device, an optical disk device, or a tape device, a flash memory or other similar solid-state memory device, or an array of devices, including devices in a storage area network or other configurations. Instructions can be stored in an information carrier. The instructions, when executed by one or more processing devices (for example, processor 302), perform one or more methods, such as those described above. The instructions can also be stored by one or more storage devices such as computer- or machine readable mediums (for example, the memory 304, the storage device 306, or memory on the processor 302). The high-speed interface 308 manages bandwidth-intensive operations for the computing device 300, while the low-speed interface 312 manages lower bandwidth-intensive operations. Such allocation of functions is an example only. In some implementations, the high speed interface 308 is coupled to the memory 304, the display 316 (e.g., through a graphics processor or accelerator), and to the high-speed expansion ports 310, which may accept various expansion cards (not shown). In the implementation, the low-speed interface 312 is coupled to the storage device 306 and the low-speed expansion port 314. The low-speed expansion port 314, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 300 may be implemented in a number of different forms, as shown in the figure. For example, it may be implemented as a standard server 320, or multiple times in a group of such servers. In addition, it may be implemented in a personal computer such as a laptop computer 322. It may also be implemented as part of a rack server system 324. Alternatively, components from the computing device 300 may be combined with other components in a mobile device, such as a mobile computing device 350. Each of such devices may include one or more of the computing device 300 and the mobile computing device 350, and an entire system may be made up of multiple computing devices communicating with each other.

The mobile computing device 350 includes a processor 352, a memory 364, an input/output device such as a display 354, a communication interface 366, and a transceiver 368, among other components. The mobile computing device 350 may also be provided with a storage device, such as a micro-drive or other device, to provide additional storage. Each of the processor 352, the memory 364, the display 354, the communication interface 366, and the transceiver 368, are interconnected using various buses, and several of the components may be mounted on a common motherboard or in other manners as appropriate.

The processor 352 can execute instructions within the mobile computing device 350, including instructions stored in the memory 364. The processor 352 may be implemented as a chipset of chips that include separate and multiple analog and digital processors. The processor 352 may provide, for example, for coordination of the other components of the mobile computing device 350, such as control of user interfaces, applications run by the mobile computing device 350, and wireless communication by the mobile computing device 350.

The processor 352 may communicate with a user through a control interface 358 and a display interface 356 coupled to the display 354. The display 354 may be, for example, a TFT (Thin-Film-Transistor Liquid Crystal Display) display or an OLED (Organic Light Emitting Diode) display, or other appropriate display technology. The display interface 356 may include appropriate circuitry for driving the display 354 to present graphical and other information to a user. The control interface 358 may receive commands from a user and convert them for submission to the processor 352. In addition, an external interface 362 may provide communication with the processor 352, so as to enable near area communication of the mobile computing device 350 with other devices. The external interface 362 may provide, for example, for wired communication in some implementations, or for wireless communication in other implementations, and multiple interfaces may also be used.

The memory 364 stores information within the mobile computing device 350.

The memory 364 can be implemented as one or more of a computer-readable medium or media, a volatile memory unit or units, or a non-volatile memory unit or units. An expansion memory 374 may also be provided and connected to the mobile computing device 350 through an expansion interface 372, which may include, for example, a SIMM (Single In Line Memory Module) card interface. The expansion memory 374 may provide extra storage space for the mobile computing device 350, or may also store applications or other information for the mobile computing device 350. Specifically, the expansion memory 374 may include instructions to carry out or supplement the processes described above, and may include secure information also. Thus, for example, the expansion memory 374 may be provide as a security module for the mobile computing device 350, and may be programmed with instructions that permit secure use of the mobile computing device 350. In addition, secure applications may be provided via the SIMM cards, along with additional information, such as placing identifying information on the SIMM card in a non-hackable manner.

The memory may include, for example, flash memory and/or NVRAM memory (nonvolatile random access memory), as discussed below. In some implementations, instructions are stored in an information carrier such that the instructions, when executed by one or more processing devices (for example, processor 352), perform one or more methods, such as those described above. The instructions can also be stored by one or more storage devices, such as one or more computer- or machine-readable mediums (for example, the memory 364, the expansion memory 374, or memory on the processor 352). In some implementations, the instructions can be received in a propagated signal, for example, over the transceiver 368 or the external interface 362.

The mobile computing device 350 may communicate wirelessly through the communication interface 366, which may include digital signal processing circuitry in some cases. The communication interface 366 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), LTE, 5G/6G cellular, among others. Such communication may occur, for example, through the transceiver 368 using a radio frequency. In addition, short-range communication may occur, such as using a Bluetooth, Wi-Fi, or other such transceiver (not shown). In addition, a GPS (Global Positioning System) receiver module 370 may provide additional navigation- and location-related wireless data to the mobile computing device 350, which may be used as appropriate by applications running on the mobile computing device 350.

The mobile computing device 350 may also communicate audibly using an audio codec 360, which may receive spoken information from a user and convert it to usable digital information. The audio codec 360 may likewise generate audible sound for a user, such as through a speaker, e.g., in a handset of the mobile computing device 350. Such sound may include sound from voice telephone calls, may include recorded sound (e.g., voice messages, music files, among others) and may also include sound generated by applications operating on the mobile computing device 350.

The mobile computing device 350 may be implemented in a number of different forms, as shown in the figure. For example, it may be implemented as a cellular telephone 380. It may also be implemented as part of a smart-phone 382, personal digital assistant, or other similar mobile device.

A number of implementations have been described. Nevertheless, it will be understood that various modifications may be made without departing from the spirit and scope of the disclosure. For example, various forms of the flows shown above may be used, with steps re-ordered, added, or removed.

Embodiments of the invention and all of the functional operations described in this specification can be implemented in digital electronic circuitry, or in computer software, firmware, or hardware, including the structures disclosed in this specification and their structural equivalents, or in combinations of one or more of them.

Embodiments of the invention can be implemented as one or more computer program products, e.g., one or more modules of computer program instructions encoded on a computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) can be written in any form of programming language, including compiled or interpreted languages, and it can be deployed in any form, including as a stand alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

In certain embodiments, processing data sets as described herein can reduce the complexity and/or dimensionality of large and/or complex data sets. A non-limiting example of a complex data set includes sequence read data generated from one or more test subjects and a plurality of reference subjects of different ages and ethnic backgrounds. In some embodiments, data sets can include from thousands to millions of sequence reads for each test and/or reference subject.

Embodiments of the invention can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the invention, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures (such as spreadsheets, relational databases, or structured files) may be used.

Particular embodiments of the invention have been described. Other embodiments are within the scope of the following claims. For example, the steps recited in the claims can be performed in a different order and still achieve desirable results.

### EXPERIMENTAL DATA

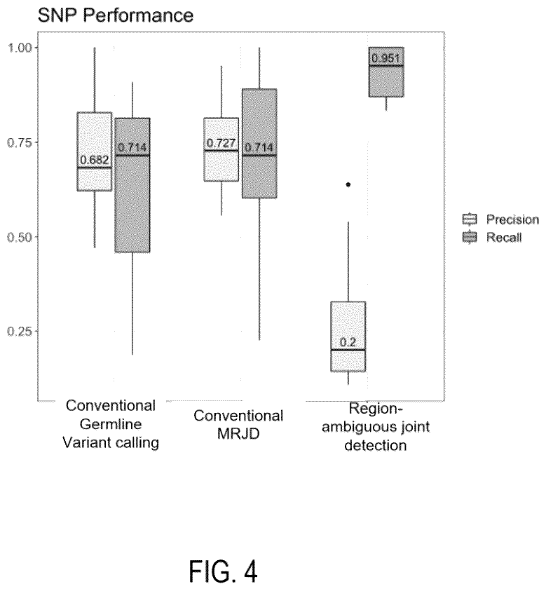

FIG. 4 is a graph showing the benchmark on conventional germline variant calling, conventional MRJD, and region-ambiguous variant detection on SNP detection in the high homology region of the PMS2 gene. The truth set was derived from LR-PCR data. The region-ambiguous variant detection enabled much greater sensitivity (i.e., 95% recall) while sacrificing precision (i.e., 20% precision), which is accepted because the goal was to identify all possible variants and do secondary testing to confirm germline pathogenic/likely pathogenic (P/LP) variant candidates.

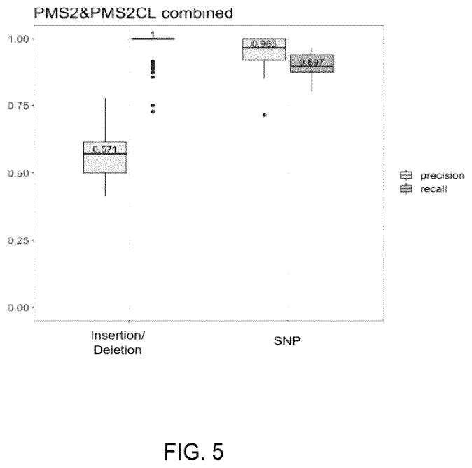

FIG. 5 is a graph showing region-ambiguous variant detection's ability to detect alternative alleles in the paralogous genes PMS2 and PMS2CL. The region-ambiguous variant detection was able to detect insertions/deletions and SNPs with 97% precision and 90% recall.

What is claimed is:

1. A method for identifying variants using region-ambiguous variant detection in genetic sample sequences, the method comprising:
obtaining a plurality of haplotypes from one or more reads of a biological sample;
generating a plurality of joint diplotype candidates comprising four or more haplotypes of the plurality of haplotypes, wherein at least two of the haplotypes of the plurality of haplotypes are associated with a first region of a reference genome and at least two other haplotypes of the plurality of haplotypes are associated with a second region of the reference genome, the plurality of joint diplotype candidates comprising multiple different copy number configurations;
determining a posterior probability for each of the plurality of joint diplotype candidates;
determining a preferred copy number configuration of the multiple different copy number configurations, wherein the preferred copy number configuration is a copy number configuration that occurs more frequently than any other copy number configuration in the plurality of joint diplotype candidates;
determining a region-ambiguous quality score for the plurality of joint diplotype candidates in a given position in a given region based on a ratio of (i) the posterior probability associated with each joint diplotype candidate of the plurality of joint diplotype candidates having the preferred copy number configuration, and (ii) the posterior probability of a homozygous joint diplotype candidate of the plurality of joint diplotype candidates that only includes the reference allele at the given position and given region of the reference genome; and
determining whether a variant exists in at least one of the first or second region of the reference genome based on the determined region-ambiguous quality score.

2. The method of claim 1, wherein the copy number configurations of the multiple different copy number configurations is a copy number of each allele in the corresponding position of interest in the first region and the second region of the reference genome.

3. The method of claim 1, wherein determining whether the variant exists in at least one of region of the reference genome based on the determined region-ambiguous quality score comprises:
comparing the region-ambiguous quality score to a predetermined threshold.

4. The method of claim 3, the method further comprising:
determining that the region-ambiguous quality score satisfies the predetermined threshold based on comparing the region-ambiguous quality score to the predetermined threshold; and
determining that an actual variant is likely to exist in at least one of the first region or the second region of the reference genome.

5. The method of claim 3, the method further comprising:
determining that the region-ambiguous quality score does not satisfy the predetermined threshold based on comparing the region-ambiguous quality score to the predetermined threshold; and
determining that an actual variant is not likely to exist in at least one of the first region or the second region of the reference genome.

6. The method of claim 1, wherein determining the region-ambiguous quality score for the plurality of joint diplotype candidates is based on the ratio of (i) the posterior probability associated with each joint diplotype candidate of the plurality of the joint diplotype candidates having the preferred copy number configuration and (ii) the posterior probability of the homozygous joint diplotype of the plurality of joint diplotype candidates that matches the reference allele in the first region of the reference genome and the second region of the reference genome comprises:
determining the region-ambiguous quality score for the plurality of joint diplotype candidates based on (i) a sum of the posterior probability associated with each joint diplotype candidate of the plurality of the joint diplotype candidates having the preferred copy number configuration and (ii) the posterior probability of the homozygous joint diplotype of the plurality of joint diplotype candidates that matches the reference allele in each of the first region of the reference genome and the second region of the reference genome.

7. The method of claim 1, wherein the first region and the second region of the reference genome are a subset of a quantity of regions of the reference genome.

8. The method of claim 7, wherein the quantity of regions of the reference genome is based on a quantity of paralogous regions located in the reference genome.

9. A system for identifying variants using region-ambiguous variant detection in genetic sample sequences, comprising:
one or more processors; and
machine-readable media interoperably coupled with the one or more processors and storing one or more instructions that, when executed by the one or more processors, perform operations comprising:
obtaining a plurality of haplotypes from one or more reads of a biological sample;
generating a plurality of joint diplotype candidates comprising four or more haplotypes of the plurality of haplotypes, wherein at least two of the haplotypes of the plurality of haplotypes are associated with a first region of a reference genome and at least two other haplotypes of the plurality of haplotypes are associated with a second region of the reference genome, the plurality of joint diplotype candidates comprising multiple different copy number configurations;
determining a posterior probability for each of the plurality of joint diplotype candidates;
determining a preferred copy number configuration of the multiple different copy number configurations, wherein the preferred copy number configuration is a copy number configuration that occurs more frequently than any other copy number configuration in the plurality of joint diplotype candidates;
determining a region-ambiguous quality score for the plurality of joint diplotype candidates in a given position in a given region based on a ratio of (i) the posterior probability associated with each joint diplotype candidate of the plurality of joint diplotype candidates having the preferred copy number configuration, and (ii) the posterior probability of a homozygous joint diplotype candidate of the plurality of joint diplotype candidates that only includes the reference allele at the given position and given region of the reference genome; and
determining whether a variant exists in at least one of the first or second region of the reference genome based on the determined region-ambiguous quality score.

10. The system of claim 9, wherein the copy number configurations of the multiple different copy number configurations is a copy number of each allele in the corresponding position of interest in the first region and the second region of the reference genome.

11. The system of claim 9, wherein determining whether the variant exists in at least one of region of the reference genome based on the determined region-ambiguous quality score comprises:
comparing the region-ambiguous quality score to a predetermined threshold.

12. The system of claim 11, the operations further comprising:
determining that the region-ambiguous quality score satisfies the predetermined threshold based on comparing the region-ambiguous quality score to the predetermined threshold; and
determining that an actual variant is likely to exist in at least one of the first region or the second region of the reference genome.

13. The system of claim 11, the operations further comprising:
determining that the region-ambiguous quality score does not satisfy the predetermined threshold based on comparing the region-ambiguous quality score to the predetermined threshold; and
determining that an actual variant is not likely to exist in at least one of the first region or the second region of the reference genome.

14. The system of claim 9, wherein determining the region-ambiguous quality score for the plurality of joint diplotype candidates is based on the ratio of (i) the posterior probability associated with each joint diplotype candidate of the plurality of the joint diplotype candidates having the preferred copy number configuration and (ii) the posterior probability of the homozygous joint diplotype of the plurality of joint diplotype candidates that matches the reference allele in the first region of the reference genome and the second region of the reference genome comprises:
determining the region-ambiguous quality score for the plurality of joint diplotype candidates based on (i) a sum of the posterior probability associated with each joint diplotype candidate of the plurality of the joint diplotype candidates having the preferred copy number configuration and (ii) the posterior probability of the homozygous joint diplotype of the plurality of joint diplotype candidates that matches the reference allele in each of the first region of the reference genome and the second region of the reference genome.

15. The system of claim 9, wherein the first region and the second region of the reference genome are a subset of a quantity of regions of the reference genome.

16. The system of claim 9, wherein the quantity of regions of the reference genome is based on a quantity of paralogous regions located in the reference genome.

17. A non-transitory computer-readable medium storing one or more instructions executable by a computer system to perform operations for identifying variants using region-ambiguous variant detection in genetic sample sequences, the operations comprising:
obtaining a plurality of haplotypes from one or more reads of a biological sample;
generating a plurality of joint diplotype candidates comprising four or more haplotypes of the plurality of haplotypes, wherein at least two of the haplotypes of the plurality of haplotypes are associated with a first region of a reference genome and at least two other haplotypes of the plurality of haplotypes are associated with a second region of the reference genome, the plurality of joint diplotype candidates comprising multiple different copy number configurations;
determining a posterior probability for each of the plurality of joint diplotype candidates;
determining a preferred copy number configuration of the multiple different copy number configurations, wherein the preferred copy number configuration is a copy number configuration that occurs more frequently than any other copy number configuration in the plurality of joint diplotype candidates;
determining a region-ambiguous quality score for the plurality of joint diplotype candidates in a given position in a given region based on a ratio of (i) the posterior probability associated with each joint diplotype candidate of the plurality of joint diplotype candidates having the preferred copy number configuration, and (ii) the posterior probability of a homozygous joint diplotype candidate of the plurality of joint diplotype candidates that only includes the reference allele at the given position and given region of the reference genome; and
determining whether a variant exists in at least one of the first or second region of the reference genome based on the determined region-ambiguous quality score.

18. The non-transitory computer-readable medium of claim 17, wherein the copy number configurations of the multiple different copy number configurations is a copy number of each allele in the corresponding position of interest in the first region and the second region of the reference genome.

19. The non-transitory computer-readable medium of claim 17, wherein determining whether the variant exists in at least one of region of the reference genome based on the determined region-ambiguous quality score comprises:
comparing the region-ambiguous quality score to a predetermined threshold.

20. The non-transitory computer-readable medium of claim 19, the operations further comprising:
determining that the region-ambiguous quality score satisfies the predetermined threshold based on comparing the region-ambiguous quality score to the predetermined threshold; and
determining that an actual variant is likely to exist in at least one of the first region or the second region of the reference genome.
